(19) United States
(12) Patent Application Publication
(10) Pub. No.: US 2012/0058564 A1
(43) Pub. Date: Mar. 8, 2012

(54) TEST KIT AND METHOD FOR MEASUREMENT OF METALS IN BIOLOGICAL FLUIDS

(76) Inventors: Karmakar Nivedita Gohil, New Delhi (IN); Manisha Sharma, New Delhi (IN); Renu Saxena, New Delhi (IN)

(21) Appl. No.: 12/998,794

(22) PCT Filed: Dec. 1, 2009
(86) PCT No.: PCT/IN2009/000696

§ 371 Date: Nov. 15, 2011

(30) Foreign Application Priority Data
Dec. 1, 2008 (IN) 2713/DEL/2008

(51) Int. Cl. G01N 21/64; G01N 33/20

(52) U.S. Cl. 436/74

(57) ABSTRACT

A test kit and a biomedical process is provided to estimate metals particularly non-transferrin bound iron levels (NTBI) in circulating body fluids particularly, serum. NTBI appears in serum when there is excess iron in the body. The method comprises of employing a signal generating moiety capable of complexing with iron that is a peptide like molecule having an iron binding site and also an optical signal generating functional group. The molecule is of microbial origin. The measurement is based on the alteration of optical characteristics of the probe molecule upon attachment of iron to its binding site on the molecule. Hence it generates a signal proportionate to the amount of iron available for binding and provides a direct estimate of free or unbound iron in the sample. According to this instant invention a rapid estimation method of NTBI in body fluids can be undertaken in an inexpensive way without the need of specialized expertise.

### FIELD OF INSTANT INVENTION

This instant invention relates to a test kit and method for measurement of metals in biological fluids. Specifically, the invention relates to a kit useful for the assay of metals such as copper, lead, zinc, iron, mercury, particularly iron in biological fluid. More specifically the invention relates to a kit useful for the measurement of iron overloads in blood serum. Still more specifically, the invention relates to a kit and method for use in pathology for direct estimation of non transferrin bound iron (NTBI) in circulating body fluids, in particular serum.

### BACKGROUND INFORMATION

Most of the metals such as copper, zinc, iron etc. when in excess, interfere with a variety of body processes, get accumulated in body tissues, and prove to be toxic to many organs and tissues including the heart, bones, intestines, kidneys, brain, and reproductive and nervous systems. Due to their hemo-concentration, serum assays of such toxic entities are generally conducted to arrive at proper therapeutic action

NTBI is a labile and potentially toxic form of serum iron associated with imbalanced iron metabolism and transfusional overload. In certain patho-physiological conditions, the iron binding capacity of transferrin is exceeded resulting in the binding of excess iron to various proteins and other putative ligands in the circulation. This excess iron which appears in the serum is collectively known as non-transferrin bound iron (NTBI). The biomedical condition is described as iron overload. NTBI is absent in healthy individuals as the serum iron is bound to an iron carrier protein transferrin.

NTBI occurs as a result of pathological conditions associated with specific diseases. Illustrative examples of such conditions include: 1) repeated transfusions, which are required by patients with various hemolytic diseases, hemoglobinopathies (among which the most common is thalassemia) or other forms of anemia whose treatment demands blood transfusions and/or iron infusion (e.g. dialysis patients) and 2) an inherited defect causing excess iron absorption, called Hereditary Hemachromatosis. Transient, reversible NTBI can also appear in the circulation of patients undergoing chemotherapy, heart bypass operations and other conditions where large amounts of iron, such as from hemoglobincatabolism, are suddenly released into the circulation. NTBI was also found in patients receiving dialysis who are treated for anemia with erythropoietin and intravenous iron supplements. Normal iron homeostasis is maintained by regulating both the absorption of iron from the diet and its distribution within the body. However, regulation of iron homeostasis is under tight control, because humans do not posses any physiological pathway for its excretion. Iron overload can be categorized as primary and secondary. Primary iron overload results from the defects in the regulation of iron balance and is best exemplified by hereditary hemochromatosis. It is characterized by excess dietary absorption of iron because of increased iron transfer from the enteral cells to the blood. Secondary iron overload, on the other hand, is acquired due to the presence of other biomedical conditions and their treatment. These are drug-induced imbalance in erythrocyte turnover (chemotherapy), chronic liver diseases, repeated blood transfusions and intravenous iron supplements. Diseases associated with secondary transfusional iron overload are β-Thalassemia (major and intermedia), sickle cell anemia, aplastic anemia and myelodysplastic syndromes.

NTBI levels vary between 1-10 μM in overload patients. It is potentially toxic because it generates free radical formation. Persistent levels of plasma NTBI leads to deposition of excess iron in tissues particularly in the liver, endocrine glands and heart, leading to various patho-physiological conditions. Thus the iron over load toxicities are the most common cause of death in patients with thalassemia due to cardiac arrest and more than 70% of adult patients suffer from hypogonadism, osteoporosis, and other endocrine disorders. About 10,000 children per year are born in India with thalassemia major trait, while the prevalence of haemochromatic disorder is 5 per 1,000 and a carrier frequency of 1 in 10. Thus, presently, iron overload is a severe, potentially fatal biomedical condition of prime concern.

In view of the damages caused by accumulation of the toxic metals, it is very important to have test kits and reliable, precise accurate methods for the estimation particularly quantification of these metals/entities, more specifically at a low concentration to enable physician to arrive at and manage appropriate therapy. Moreover, such tests should be simple, cost effective, time effective, easily accessible and affordable to common people. With special reference to iron overload, NTBI-screening might be more valuable to diagnose individuals with hereditary hemachromatosis, who do not show any symptoms at early stage and get misdiagnosed due to very low level of transferrin-iron saturation.

The prior art with respect to test kit known to the inventor includes WO 2008046086 titled ‘Instruments for direct detection of free metals in fluids and methods to diagnose metal-related diseases and determine pharmacologic dosing regimens’ to Pipex, Inc., discloses an apparatus and method for measurement of copper in serum. The claimed Apparatus for measuring free copper levels in blood, comprising: a multifunctional filter for creating filtrate from a blood sample by filtering out particles which would interfere with measurement of free copper in the blood sample and conditioning the filtrate to an appropriate pH to allow current flow through the particles including particles larger than about 130 kD and particles having copper bound thereto; a detector apparatus including electrodes for detecting current flow through the filtrate; a display for displaying the free copper level in the blood sample based on the current flow detected between the electrodes. Thus it is apparent that the invention uses potentiostat for detecting copper in both free and bound form. Further, it also discloses, though not claimed, that the apparatus can be used for determination NTBI. As such, the description does not give any clue for further research to improve the instrument.
The major drawback is that the instrument appears to be cost extensive requires infrastructure, skilled personnel and stringent operating conditions. The description also fails to substantiate use of potentiostat for measuring NTBI.
The literature available with regard to measuring of serum lead, indicates that lead also can be evaluated by measuring erythrocyte protoporphyrin (EP) in blood samples. [Patrick, L (March 2006). “Lead toxicity, a review of the literature. Part 1: Exposure, evaluation, and treatment”. Alternative medicine review 11 (1): 2-22. ISSN 1089-515918]. EP is a part of red blood cells known to increase when the amount of lead in the blood is high. However, the EP level alone is not sensitive enough to identify elevated blood lead levels below about 35 μg/dL. Due to this higher threshold for detection and the fact that EP levels also increase in iron deficiency, use of this method for detecting lead exposure has decreased. Further, blood lead levels are an indicator mainly of recent or current lead exposure, not of total body burden. Lead in bones can be measured non-invasively by X-ray fluorescence; this may be the best measure of cumulative exposure and total body burden. However this method is not widely available and is mainly used for research rather than routine diagnosis. As such no fool-proof method is available.
Moreover, lead unlike Iron or Copper can be easily complexed with chelators such as EDTA and removed from the body conveniently. Similar analogy also works with mercury. Methyl mercury complex can be formed to throw mercury out of the body.
U.S. Pat. No. 4,224,034 titled Assay of iron and iron binding protein reagents and methods discloses a process for detecting the presence of ferrous ions employing 9-(2-pyridyl)-acenaphtho[1,2-e]-as-triazine as both a chelator and indicator of ferrous ion.
PCT/IL99/00677 filed on Dec. 13, 1999 and PCT/IL01/00384 filed on Apr. 29, 2001 relate to a ‘Method for measuring non-transferrin bound iron. PCT/IL99/00677 also claims a kit for determination of the concentration of a non-bound metal ion in a sample of serum or other biological fluids. The claimed kit comprises a multi well plate coated with a polymer conjugated form of metal chelator selected from desferrioxamine (DFO).
The claimed method comprises:
(i) providing a surface coated with a polymer conjugated form of a metal chelator,
(ii) bringing said sample into contact with said coated surface for a period of sufficient time to allow the metal ion to be captured by the metal chelator,
(iii) bringing in to contact with said coated surface after step (ii) a marker conjugated with a moiety that can be captured by the metal chelator,
(iv) determining the marker that has been released by capture of metal ion by the coated surface and
(v) the concentration of metal ion in the sample is calculated from the binding sites left available for capturing the metal ion bound to the marker.
   The process being multi-step is labor intensive and less precise. Further it also involves incorporation of an additional marker conjugated moiety.
   PCT/IL01/00384 discloses improved process for measuring non-transferrin bound iron. The process claims to overcome the drawbacks of the process disclosed in copending application numbered PCT/IL99/00677 by reducing the process steps to two. Each stage comprises contacting the sample with a reagent and measuring the fluorescence of the contacted sample, wherein in the first stage the reagent is a fluorescent probe and in the second stage the reagent is a fluorescent probe with the addition of a large amount of a Fe chelator. The fluorescent probe that is used in the second stage may be the same that is used in the first stage and may comprise a fluorescent marker combined with a Fe chelator. The fluorescent marker may be fluorescein, or derivatives of fluorescein, coumarin and BODIPY.
   The main problem faced by this process is fluorescent tagging of a molecule. The fluorescent tagging of a molecule is a cumbersome process and sometimes the quantum yield may not be high because of which the signal generated is weak.
   According to the disclosure in the specification of PCT/IL99/00677, the presently available Prior Art in respect of diagnostic methods are limited in scope, and are basically divided into two groups: Detection of iron-overload: Three routine clinical tests are available for detecting excess iron in the circulation: 1. total serum iron by chemical or physicochemical methods, 2. percent transferrin-iron saturation, or serum iron-binding capacity, by measuring high-affinity binding of radioactive iron to serum components essentially transferrin) and 3. circulating ferritin levels by immunoassay. Although these three indicators tend to be elevated in most cases of severe iron-overload, they often fail to detect lower iron-load levels and can also fluctuate for reasons unrelated to iron-status. The most commonly used of these tests is circulating ferritin levels, even though its diagnostic value for iron-status is controversial and can even be misleading in some cases. Since excess body iron accumulates first in the liver, analysis of liver biopsies constitutes a definitive diagnosis of iron-overload disease.
   Detection of NTBI: There are two main methods for NTBI determination currently used in research laboratories. However, because of their drawbacks, as explained below, they are not in routine clinical use.
   One methodology was originally developed by Hershko and coworkers [Hershko, H., Graham, G., Bates, G. W., and Rachmilewitz (1978) British J. Haematol. 40, 255-263] and later refined by Singh and coworkers [Singh, S., Hider, R. C. and Porter, J. B. (1990) Anal. Biochem. 186, 320-323]. In brief, the refined method is as follows: Step 1. A serum sample (1 ml) is mixed with 80 mM nitrilotriacetic acid (to solubilize the NTBI); Step 2. The sample is filtered by centrifugation on Centricon filters with a 25 kD molecular weight cut-off; Step 3. The protein-free filtrate is injected into an HPLC column derivatized with the iron chelator deferriprone (or Ll), which forms a stoichiometric coloured complex with iron giving a quantitative value of the amount of iron in the sample.
   The three main drawbacks of this method are its cost, its cumbersome nature, which makes it difficult to set up in non-specialized laboratories, and its relatively low throughput efficiency.
   A second method [Evans, P. J. and Halliwell, B. (1994) Methods Enzymol., 233, 82-89] employs the antibiotic bleomycin, which combines with NTBI, but not with transferrin-bound iron, to form highly reactive complexes which generate DNA cleavage products. The relative amount of DNA cleavage products is proportional to the amount of input NTBI and is quantified by the thiobarbituric acid test. The drawback of this method is that it tends to overestimate NTBI and may give false positive results.
   As herein before described, Iron overload is diagnosed only indirectly by estimating total serum iron, percent transferrin saturation and transferrin iron binding capacity by physicochemical methods and also by determining the serum ferritin levels by immunoassay. Although these methods are quite effective in detecting severe iron overload, they fail to detect low to moderate levels of iron overload. Further, studies have shown that in hemochromatosis patients NTBI is present, in spite of incomplete transferrin saturation. Therefore these classical parameters may not be indicative of an accurate picture of the iron status of patients. For this reason it is important to monitor and accurately quantify this potentially toxic iron fraction.

As described in PCT/IL99/00677, there is no generally accepted routine biomedical assay for the accurate quantification of NTBI particularly at low concentration.

At the research level, few methods exist for quantification of NTBI, such as high-performance liquid chromatography (HPLC) [Singh, S., Hider, R. C. and Porter, J. B. (1990) Anal. Biochem. 186, 320-323] or inductive conductiometric plasma spectrometry (ICP) [Gosriwatana, I., Loreal, O., Lu, S., Brissot, P., Porter, J. and Hider, R. C. (1999) Anal. Biochem. 273, 212-220]. Although these detection methods have high reliabilities, they are very labor intensive and are difficult to set up in non-specialized laboratories. Also, methods employing iron-sensitive fluorescence probes have been reported, such as fluorescein-labeled desferoxamine (Fl-DFO) and fluorescein-labeled apotransferrin (Fl-aTf), to quantify NTBI in 96-well plate set up [Breuer, W. and Cabantchik, Z. I. (2001) Anal. Biochem. 299, 194-202]. A limitation of these methods is their tendency to be affected by conditions of the local environment such as serum color and turbidity and thereby may give false positive results.

It is apparent from the disclosure herein above, that no test kits are commercially available for the direct measurement of metals such as copper, NTBI of prime significance, with regard to toxic effects, from biological fluids or serum at lower concentration. Further, no well accepted precise method of economic significance is available for routine biomedical assays for quantification of such metals preferably NTBI.

In view of the above scenario, there is a widely recognized need, to develop a precise, reproducible, rapid, simple, economic biomedical method requiring easily available reagents and a test kit for the estimation and quantification of NTBI devoid of limitations associated with existing technology. A highly sensitive process for quantification of NTBI that is, cost effective, providing high throughput efficiency, without compromising accuracy and sensitivity and having broad biomedical application in both the identification and validation of treatment regimens for iron overload conditions is today's need to meet socioeconomic demands.

After prolonged R & D the inventors have found out that the application of naturally occurring intrinsically fluorescent compound with metal complexing ability of microbial origin can be the effective analytical tool for detecting and quantifying the desired metals from biological fluids. Though, the test kit can be applicable for detection and quantification of most of the metals which become toxic on accumulation, in biological fluids with appropriate deviation, it is illustrated with reference to NTBI. Fluorescent siderophores secreted by certain microbes under iron deprived conditions, fall in this category and have high affinity (K=10²⁰-10⁵²) for iron. The combined property of metal chelating/complexing and fluorescence emission gives added advantage to such compounds. Typically, they are low molecular weight (MW ca. 400-2000 Da) aqueous soluble organic ligands functioning as metal/iron chelators and sequester it from the organism's immediate environment. This instant invention describes the development of a biomedical method to estimate NTBI in biological fluids using a siderophore and a kit therefor.

The developed kit and a method prove to be versatile, economical, sensitive and of a high throughput nature. The method is precise, highly sensitive and can detect 0.07 μM of NTBI.

The main object of the present invention is to provide a test kit and method for measurement of metals in biological fluids substantially obviating the drawbacks of existing technology.
Other object is to provide kit and method useful for the assay of metals such as copper, zinc, iron, mercury, particularly iron in biological fluid.
Another object is to provide kit and method useful for the measurement of iron overloads in blood serum.
Still another object is to provide kit and method for direct estimation and quantification of Non Transferrin Bound Iron (NTBI) in circulating body fluids, in particular serum.

### STATEMENT OF INVENTION

Accordingly, the present invention provides a test kit useful for the assay of excess metals in biological fluids comprising:
(i) a reagent capable of blocking free metal binding sites of respective protein,
(ii) agent capable of releasing metal ions bound to ligands other than the respective protein
(iii) separation means to obtain protein-free solution,
(iv) signal generating moiety capable of complexing with metal to be detected and developing signals
(v) means to measure and display signals.
   According to one of the embodiments, the present invention provides a test kit useful for the assay of NTBI from biological fluids comprising:
(i) a reagent capable of blocking free iron binding sites of transferrin protein,
(ii) agent capable of releasing iron bound to ligands other than transferrin
(iii) separation means to obtain protein-free solution,
(iv) signal generating moiety capable of complexing with iron to be detected and developing signals
(v) means to measure and display signals.
   According to other aspect of the present invention there is provided a method for estimation/detection/quantification of metals in biological fluids comprising of the steps:
(i) obtaining a sample of the biological fluid of the subject,
(ii) contacting the metal containing sample with a reagent capable of blocking free metal binding sites of respective protein,
(iii) subsequently releasing metal ions bound to ligands other than the respective protein by contacting the solution obtained from step (ii) with releasing agent,
(iv) separating dissolved proteins to obtain protein-free solution,
(v) contacting the protein-free solution obtained in step (iv) with signal generating moiety capable of complexing with metal to be detected followed by detecting and quantifying said signal thereby quantifying metal levels in the biological fluid.
   The reagent capable of blocking free metal binding sites of respective protein may be such as other metals preferably belonging to same group of periodic table.
   According to a preferred embodiment, there is provided a method for estimation/detection/quantification of NTBI in biological fluids comprising of the steps:
(i) obtaining a sample of the biological fluid of the subject,
(ii) contacting the said sample with a reagent capable of blocking free iron binding sites of transferrin protein,
(iii) subsequently releasing iron bound to ligands other than transferrin by contacting the solution obtained from step (ii) with releasing agent,
(iv) separating dissolved proteins to obtain protein-free solution,
(v) contacting the protein-free solution obtained in step (iv) with signal generating moiety capable of complexing with iron to be detected followed by detecting and quantifying said signal, thereby quantifying NTBI levels in the biological fluid
   According to preferred embodiment of this invention, the biological fluid used for detection of NTBI may be such as blood serum.
   The reagent capable of blocking free iron binding sites of transferrin protein employed in step (ii) may be such as salts of trivalent transition metals exemplified by cobalt and gallium or myeloperoxidase system preferably cationic salts of cobalt, more preferably citrate complex of cobalt (III).
   The contacting in step (ii) may be effected for 15-30 minutes at ambient temperature preferably between 35° C. and 40° C.
   In a preferred embodiment, wherein the releasing agent used may be such as moderate Fe³⁺ chelators comprising the group consisting of EDTA, sodium-oxalate, or nitrilotriacetate preferably sodium-oxalate, or nitrilotriacetate more preferably, aqueous solution of Nitrilotriacetic acid disodium salt (NTA).
   The releasing may be carried out at physiological pH employing preferably NTA at concentration of 800 mM.
   Separation to obtain a protein-free solution may be carried out by employing ultra filter preferably the one having a molecular weight cut-off of 10-30 Kda.
   Signal generating moiety capable of complexing with NTBI employed in step (v), and developing signals may be an intrinsically fluorescent compound, a peptide of microbial origin such as a siderophore/fluorophore.
   The siderophore/fluorophore used may be azotobactin the one secreted by genus Azotobacter or pyoverdine the one secreted by Pseudomonas aeruginosa preferably azotobactin.
   Azotobactin can be obtained by culturing readily available Azotobacter genus by conventional methods. The culturing process is simple leading to good yield of azotobactin which can be produced in bulk, purified, lyophilized and then stored over long periods under refrigeration for future use. Further, the cost of culture and purification is a lot less than employing synthetic fluorochrome tagged to complexing moiety, because fluorescent dyes are expensive.
   Contacting with azotobactin may be conducted at least for 10 minutes preferably till the fluorescence reading gets stabilized and using 1 μM solution in acetate buffer of pH 4-6. Detecting and quantifying the signal so generated may be carried out by measuring fluorescence at 490 nm using conventional spectrofluorimeter.

### BRIEF DESCRIPTION OF DRAWINGS

FIG. 1: A block diagram of the test kit.

FIG. 2: Depicts Schematic of method protocol

FIG. 3: Relates to a calibration curve constructed with a series of known iron concentration in serum of normal controls

FIG. 4: Graph illustrating a linear correlation between NTBI levels and Percent transferrin saturation in biomedical samples.

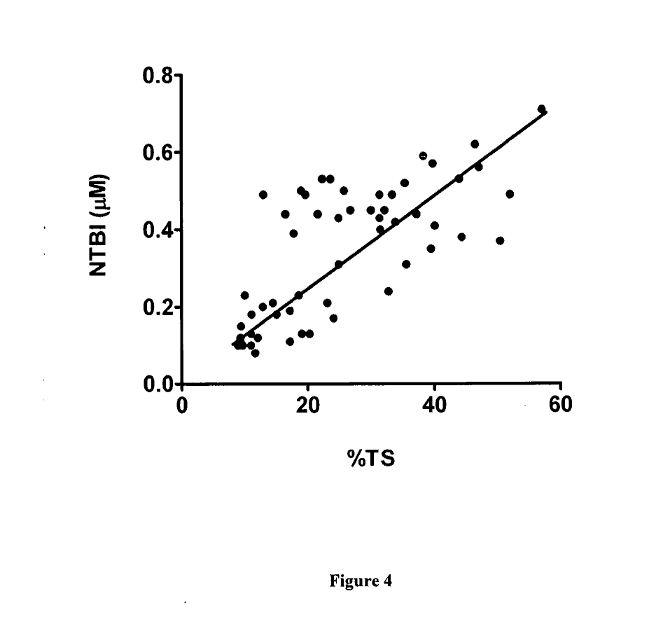

### DESCRIPTION OF THE INVENTION

The invention relates to a test kit and a method for measurement of iron overloads in biomedical processes. More particularly, the invention relates to a kit and assay method for use in pathology for direct estimation of non transferrin bound iron (NTBI) in circulating body fluids, in particular serum. NTBI is a labile and potentially toxic form of serum iron associated with imbalanced iron metabolism and transfusional overload.
A preferred embodiment of the instant invention discloses an assay process for accurate estimation/quantification of NTBI in circulating body fluids preferably serum, specifically at low concentration. Further, the process is precise, reproducible, cost effective, environmentally safe, industrially feasible, and does not require any stringent operating conditions.
In the instant invention the biomedical method provided for the estimation of NTBI in circulating body fluids, comprising of a detection probe which has an iron binding moiety, and also a signal generating moiety. The intensity of generated signal is related to an amount of the iron bound to the detection probe.
In the instant invention serum is separated from the circulating body fluids and kept at −20° C. until analysis.
In the instant invention, a releasing/mobilizing agent is added to the above mixture, and is allowed to stand for 15-30 minutes at 35° C.-40° C.
In the instant invention the above mixture is filtered through an ultrafilter and the ultrafiltrate is collected.
In the instant invention, to a known volume of ultrafiltrate is added a fixed quantity of measuring solution and allowed to stand for 5-20 minutes at 30° C.-35° C.
In the instant invention, fluorescence intensity of the mixture described in above claim is measured in spectrofluorimeter.
In a preferred embodiment the circulating body fluid is blood, serum or plasma.
In a preferred embodiment the blocking agent cobalt in the form of citrate complex of cobalt(III) derived from chloride salt is efficient blocking agent in the stated experimental conditions.
In a preferred embodiment herein the citrate complex of cobalt(III) does not alter the fluorescent characteristics of the detection probe in the stated experimental conditions.
In a preferred embodiments, wherein the releasing/mobilizing agent is selected from the group consisting of sodium-oxalate and nitrilotriacetate
In a preferred embodiments, ultrafilter used has a molecular weight cut-off of 10-30 Kda.
In a preferred embodiment, the signal generating moiety is a fluorophore and also has ability to form chelation complex with NTBI.
In a preferred embodiment the intensity of signal is stoichiometrically related to the iron bound by the iron binding moiety of detection probe.
In a preferred embodiment the method compares the signal generated from the sample to the signal generated from blank.
In a preferred embodiment the signal generated is quantified by using a calibration curve; the calibration curve depicting a fluorescence quenching against known iron concentration in the circulating body fluids.
Accordingly, it will thus be seen from the foregoing description of the invention according to the embodiments of the invention herein set forth, that the present invention provides a new test kit useful to assay primarily for serum metal particularly iron determination, and provides a novel and advantageous method and reagents therefor, all having desired advantages and characteristics, and accomplishing the objects of the invention including the objects herein before pointed out and others, which are inherent in the invention.
It will be understood that certain modifications and variations of the specific and general concepts of the invention may be effected without departing from the many concepts heretofore described; accordingly, the invention is not to be considered limited to the specific form or embodiments set forth herein for the purpose of disclosing and illustrating the inventive concepts discovered and herein applied. For example, although the present assay system dwells primarily on the determination of NTBI in serum, the principles and concepts set forth would apply advantageously to the determination of other metals with similar behaviour. The invention is further illustrated by the following examples, which do not construe the scope of the claimed protection.

### EXAMPLES

### Example 1

### Method to Estimate NTBI Levels in Serum

### Chemicals:

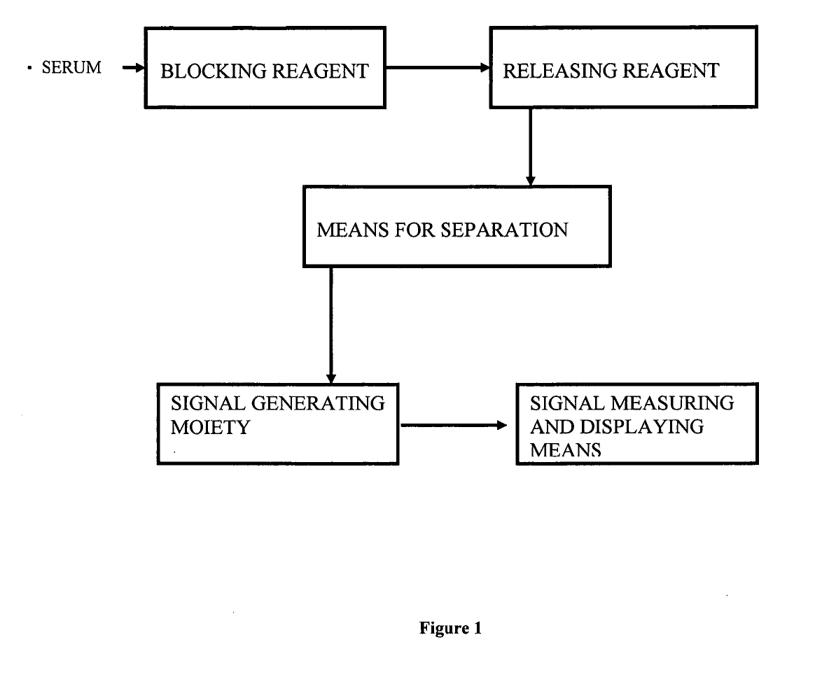

Blocking agent reagent: The reagent was prepared by dissolving Cobalt(II) chloride hexahydrate (100 mM) in 1.2M citrate buffer (pH 5-6) including 40 mM peroxide solution. Further dilutions were done in deionized water to obtain a final concentration of 5 mM.
Releasing/Mobilizing reagent: Nitrilotriacetic acid disodium salt (NTA) was dissolved in deionized water to obtain a concentration of 800 mM (pH 7-7.2).
Measuring solution: A purified siderophore solution (1 μM=0.03 A₃₈₀) prepared in acetate buffer (pH 4-6), was used for quantification of NTBI.
Method protocol:
The procedural flow chart of the developed process is schematized in the FIG. 1. Serum obtained from blood sample and blocking agent stock solution were mixed in the ratio of 4.5:1 respectively. The contents are vortexed for proper mixing and incubated for 30 minutes at 37° C. Mobilizing agent was then added to the serum mixture (20% V/V). This reagent mixture was allowed to stand for next 30 minutes at room temperature. The serum mixture was then filtered (10-30 Kda cut off). Ultrafiltrate and measuring solution were mixed (1:30), in disposable fluorescence cuvette and allowed to stand for 10 minutes. The fluorescence intensity of this solution was measured with luminescence spectrometer LS 50B (Perkin Elmer, UK) at λ_{exc}/λₑₘ 380 nm/490 nm.

### Example 2

### Construction of Calibration Curve

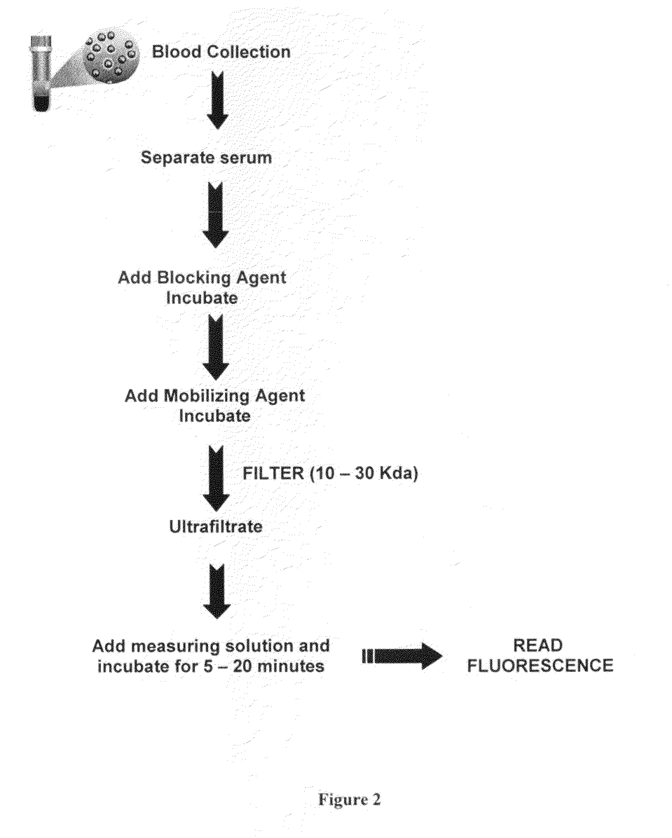

In order to determine the sensitivity of the method and to provide a reference curve for the estimation of unknown serum samples from patients, the procedure described in Example 1 was conducted on serial dilutions of known concentrations of standard iron (E-Merck Germany). The concentration range used for the construction of calibration curve is 0-0.7 μM. Whole blood was obtained from normal subjects (healthy volunteers with informed consent) and serum extracted out of it. A plot of the ratio of fluorescence intensity (F₀/F) versus input iron concentration is generated. F₀ and F are the fluorescence intensities at a maximum of emission in the absence and presence of standard iron solution respectively. The results are depicted in FIG. 2. The best fit was obtained by nonlinear regression analysis using the exponential association model.

### Example 3

### A Method to Estimate NTBI Levels in Blood Serum of β-Thalassemia Major Patients Undergoing Chelation Therapy

The reagents and method are essentially as described for Example 1 herein above. Serum samples were collected from 63 patients suffering from β-Thalassemia major. 42 males and 21 females between the age of 2 to 25 years (12.36±5.43 years; mean±SD) were selected. The patients were receiving regular blood transfusion and were under chelation therapy. Sera were separated within 1 hr of collection to avoid the possible release of iron from hemolysis of erythrocytes. The serum samples were stored at −20° C. until the time of analysis. The NTBI levels obtained with the present method ranged from 0.07-3.24 μM (0.44±0.16 μM; mean±SD). This method was found to be highly sensitive with very low detection levels compared to earlier methods described in the literature.

Comparison of NTBI measurements were done in 10 thalassemic serum samples, between the present method and the established colorimetric bathophenanthroline (BPT) method as illustrated in Table 1. The latter underestimates NTBI levels possibly because of incomplete conversion of ferric ions to ferrous ions which forms the basis of that estimation procedure.

TABLE 1

| Serum | NTBI Levels (μM) | BPT |
|---|---|---|
| Sample | Present Method | Method |
| 1 | 0.490 | 0.183 |
| 2 | 0.260 | 0.106 |
| 3 | 0.070 | 0.000 |
| 4 | 3.240 | 1.540 |
| 5 | 0.500 | 0.390 |
| 6 | 0.200 | 0.102 |
| 7 | 0.110 | 0.000 |
| 8 | 0.410 | 0.222 |
| 9 | 0.710 | 0.450 |
| 10 | 1.120 | 0.873 |

### Example 4

### Correlation Between the NTBI Levels and Transferrin Saturation in β-Thalassemia Major Patients

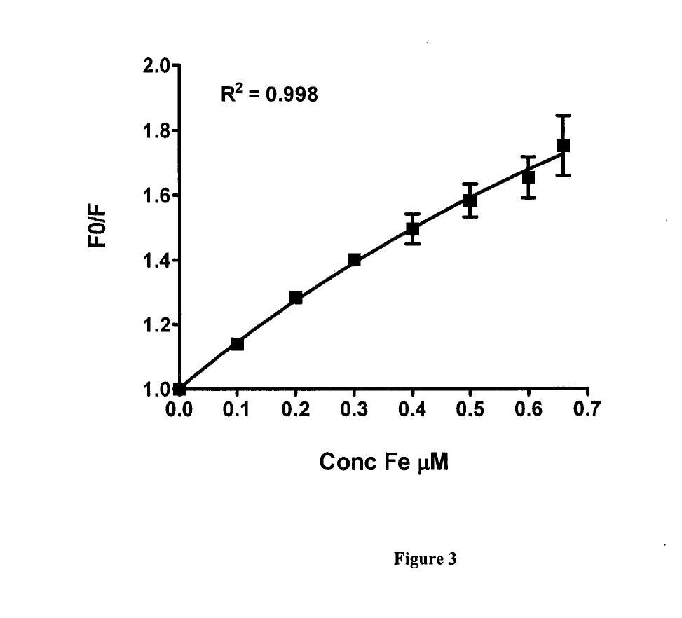

A group of 56 β-Thalassemic major patients, who were undergoing chelation therapy were tested for transferrin saturation (% TS) and NTBI levels. NTBI levels were determined by the present method describe in Example 1. As shown in FIG. 3 there is a significant correlation between the two parameters, with Pearson coefficient equal to 0.73 and p value less than 0.0001.

We claim:

1. A test kit useful for the assay of excess metals from biological fluids comprising:
(i) a reagent capable of blocking free metal binding sites of respective protein,
(ii) agent capable of releasing metal ions bound to ligands other than the respective protein
(iii) separation means to obtain protein-free solution,
(iv) signal generating moiety capable of complexing with metal to be detected and developing signals,
(v) means to measure and display signals.

2. A test kit as claimed in claim 1 useful for the assay of non transferrin bound iron (NTBI) from biological fluids comprising:
(i) a reagent capable of blocking free iron binding sites of transferrin protein,
(ii) agent capable of releasing iron bound to ligands other than transferrin,
(iii) separation means to obtain protein-free solution,
(iv) signal generating moiety capable of complexing with iron to be detected and developing signals
(v) means to measure and display signals.

3. A test kit as claimed in claim 1 wherein the excess metal assayed comprising copper, zinc, iron, lead, mercury.

4. A test kit as claimed in claim 1 wherein the excess metal assayed is NTBI.

5. A test kit as claimed in claim 2 comprising:
(i) trivalent transition metals exemplified by cobalt and gallium,
(ii) iron chelator comprising EDTA, sodium-oxalate, or nitrilotriacetate preferably aqueous solution of Nitrilotriacetic acid disodium salt (NTA),
(iii) ultra filter,
(iv) intrinsically fluorescent compound of microbial origin, and
(v) spectrofluorometer.

6. A method for estimation/detection/quantification of metals in biological fluids using a kit as claimed in claim 1 comprising of the steps:
(i) obtaining a sample of the biological fluid of the subject,
(ii) contacting the metal containing sample with a reagent capable of blocking free metal binding sites of respective protein,
(iii) subsequently releasing metal ions bound to ligands other than the respective protein, by contacting the solution obtained from step (ii) with releasing agent,
(iv) separating dissolved proteins to obtain protein-free solution,
(vi) contacting the protein-free solution obtained in step (iv) with signal generating moiety capable of complexing with metal to be detected followed by detecting and quantifying said signal thereby quantifying metal levels in the biological fluid.

7. A method for estimation/detection/quantification of NTBI in biological fluids using a kit as claimed in claim 2 comprising of the steps:
(i) obtaining a sample of the biological fluid of the subject,
(ii) contacting the said sample with a reagent capable of blocking free iron binding sites of transferrin protein,
(iii) subsequently releasing iron bound to ligands other than transferring, by contacting the solution obtained from step (ii) with releasing agent,
(iv) separating dissolved proteins to obtain protein-free solution,
(v) contacting the protein-free solution obtained in step (iv) with signal generating moiety capable of complexing with iron to be detected followed by detecting and quantifying said signal thereby quantifying NTBI levels the biological fluid.

8. A method as claimed in claim 7 wherein, the biological fluid used for detection of NTBI is blood serum.

9. A method as claimed in claim 7 wherein the reagent capable of blocking free iron binding sites of transferrin protein employed in step (ii) is salts of trivalent transition metals exemplified by cobalt and gallium or myeloperoxidase system preferably cationic salts of cobalt, more preferably citrate complex of cobalt (III).

10. A method as claimed in claim 7 wherein, the contacting in step (ii) is effected for 15-30 minutes at ambient temperature preferably between 35° C. and 40° C.

11. A method as claimed in claim 7 wherein, the releasing agent used is a moderate Fe³⁺ chelator comprising the group consisting of EDTA, sodium-oxalate, or nitrilotriacetate preferably sodium-oxalate, or nitrilotriacetate more preferably, aqueous solution of Nitrilotriacetic acid disodium salt (NTA).

12. A method as claimed in claim 7 wherein, the releasing is carried out at physiological pH employing preferably NTA at concentration of 800 mM.

13. A method as claimed in claim 7 wherein, the separation to obtain a protein-free solution is conducted by employing ultra filter preferably the one having a molecular weight cut-off of 10-30 Kda.

14. A method as claimed in claim 7 wherein, the signal generating moiety capable of complexing with NTBI employed in step (v), and developing signals is an intrinsically fluorescent compound, a peptide of microbial origin such as a siderophore/fluorophore.

15. A method as claimed in claim 14 where in the siderophore/fluorophore is azotobactin, the one secreted by genus Azotobacter or pyoverdine the one secreted by Pseudomonas aeruginosa preferably azotobactin.

16. A method as claimed in claim 7 wherein the contacting with signal generating moiety is conducted at least for 10 minutes preferably till the fluorescence reading gets stabilized and using 1 μM solution in acetate buffer of pH 4-6.

17. A method as claimed in claim 7 wherein the detecting and quantifying the signal so generated is carried out by measuring fluorescence at 490 nm using conventional spectrofluorimeter.

18. A method as claimed in claim 7 comprises of the following steps:
(i) obtaining blood serum,
(ii) contacting the said serum with citrate complex of cobalt (III) up to 30 minutes, followed by
(iii) contacting with aqueous solution of Nitrilotriacetic acid disodium salt (NTA) at physiological pHto get NTBI released,
(iv) subjecting to ultra fitration employing filter having a molecular weight cut-off of 10-30 Kda to procure protein free solution,
(vi) contacting the protein free solution so obtained with azotobactin secreted by genus Azotobacter in acetate buffer of pH 4-6 at least for 10 minutes, till the fluorescence reading gets stabilized,
(vii) measuring fluorescence at 490 nm and,
(viii) quantifying NTBI with pre calibrated curve.

19. The results of bioassay obtained by the test kit as claimed in claim 2 is useful for the assay of excess metals preferably NTBI from biological fluids are useful to arrive at therapeutic treatment for managing diseases associated with iron overload in circulating biological fluids.

20. The method as claimed in claim 7 is capable of detecting NTBI is blood serum as low as) 07 μM.
